(19) United States
(12) Patent Application Publication
(10) Pub. No.: US 2004/0186181 A1
(43) Pub. Date: Sep. 23, 2004

(54) Method and composition for decreasing ghrelin levels

(75) Inventors: Debasis Bagchi, Concord, CA (US); Shil Kothari, Fairfield, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(73) Assignee: InterHealth Nutraceuticals, Incorporated, Benicia, CA

(21) Appl. No.: 10/805,129

(22) Filed: Mar. 19, 2004

Related U.S. Application Data
(60) Provisional application No. 60/456,592, filed on Mar. 21, 2003.

(51) Int. Cl. A61K 31/19

(52) U.S. Cl. 514/574

(57) ABSTRACT

A method and composition for reducing ghrelin levels in a person or other mammal. The method involves administration of a composition incorporating (−) hydroxycitric acid in an amount sufficient to decrease the ghrelin levels in a person or other mammal. The composition comprises (−)hydroxycitric acid in an amount sufficient to decrease the ghrelin levels in the person or other mammal. The composition of the method and related composition can also incorporate gymnemic acid, green tea extract, and oxygen coordinated niacin-bound chromium. The method and related composition are effective in reducing ghrelin levels in a person or mammal, which decreases feelings of hunger in a person or other mammal and allow for effective weight management.

This application claims the benefit of U.S. Provisional Application No. 60/456,592 filed Mar. 21, 2003.

### BACKGROUND Of THE INVENTION

The present invention relates generally in a method and related composition for decreasing ghrelin levels in a person or other mammal who would benefit from such a reduction and more particularly to a method and composition incorporating (−)hydroxycitric acid (HCA) to decrease ghrelin levels in a person or other mammal.

Ghrelin is a 28-amino acid gastric acylated peptide and an endogenous ligand for the growth hormone seretagogue receptor (GHS-Rs). It regulates secretion of pituitary growth hormone (GH), as well as GHS-Rs distributed in hypothalamic neurons and in the brainstem. Increased levels of ghrelin have been demonstrated to increase food intake in rodents and humans. Ghrelin has been shown to participate in energy balance in rodents by decreasing fat utilization, without significantly changing energy expenditure or locomotor activity of the rodents. Ghrelin, as a brain-gut peptide, also is involved in regulation of food intake and body weight in persons.

A known biomarker for genetic propensity of a person toward obesity is serum leptin, a hormone encoded by the gene that regulates body weight and body mass index. Leptin binds to receptors in the brain, where it activates signals that inhibit food intake and increase energy expenditure. Studies have shown that plasma leptin levels are higher in overweight than in non-overweight individuals, and higher in women than in men. A relationship has been established between characteristic changes in serum ghrelin levels and serum leptin levels in obese human subjects. Specifically, increases in ghrelin levels are related to increases in serum leptin levels. It also has been established that ghrelin influences food intake through altering levels of neuropeptide Y (NPY). Increases in ghrelin lead to increases in NPY MRNA, whereby increasing the translation rate and release of NPY and thus inducing orexigenic (i.e. induced feeding) behavior. Plasma ghrelin levels rise sharply shortly before and fall shortly after every meal in obese subjects, and, as stated above, plasma ghrelin and plasma leptin levels are correlated.

Increases in leptin stimulates the anorexigenic (i.e. loss of appetite) pathway by stimulating α-melanocyte-stimulating hormone (α-MSH) as well as inhibit NPY production via NPY/Agouti-related neuron down regulation. In a homeostatic state, ghrelin and leptin work to balance orexigenic and anorexigenic states by regulating and ensuring NPY release is controlled thus preventing orexigenic or anorexigenic behavior. However, in overweight individuals serum leptin and ghrelin levels are elevated. Since leptin decreases NPY and ghrelin increases NPY production, it is logical to ascertain that they would cancel each other out and the net result would be a homeostatic state. However, this is not the case because of leptin resistance, a conditioned associated with increased weight, in which leptin can not bind to the NPY/Agouti-related neuron due to poor binding to the receptor. Ghrelin can bind freely to NPY/Agouti-related neurons creating a large increase in NPY production and thus leading to orexiginic behavior. Besides the above-discussed relationship to feeding behavior, body weight and body mass index, ghrelin levels also are related to control of total blood glucose, cholesterol and triglyceride levels, blood sugar control, and also to gastric protection, such as prevention of acid reflux and gastric ulcers in persons.

Therefore, it should be appreciated that a need exists for a method and composition that reduces ghrelin levels to decrease and regulate food intake, increase fat metabolism and provide other additional benefits associated with maintaining health body weight in humans and other mammals. The present invention fulfills these needs and provides further related advantages.

### SUMMARY OF THE INVENTION

The present invention resides in a method and composition for reducing ghrelin levels, the reduction of which increases fat oxidation, decreases and regulates food intake, and provides additional benefits related to maintaining a healthy body weight. The method involves administering a composition including (−)hydroxycitric acid to the person or other mammal in an amount sufficient to decrease ghrelin levels in that person or other mammal.

The method and related composition of the present invention are effective at reducing ghrelin levels in people and other mammals. Reduction of ghrelin levels results in a time-dependent decrease in appetite, increase in fat oxidation, decrease and regulation of food intake, and additional benefits related to maintaining a healthy body weight in people and other mammals.

More particularly, in one aspect of the invention, the method of the present invention involves administering to a person or other mammal a composition comprised of hydroxycitric acid in an amount that is sufficient to decrease ghrelin levels in that person or other mammal to which the composition was administered.

In a further aspect of the invention, the method of the present invention involves administering to a person or other mammal in need of reducing or maintaining body weight a composition comprised of hydroxycitric acid in an amount sufficient to decrease ghrelin levels in the person or other mammal to which the composition was administered.

In a further aspect of the present invention, the hydroxycitric acid is bound to one or more metals to form a single, double or triple salt

In a further aspect of the present invention, the metal or metals the hydroxycitric acid is bound to from a single double or triple salt are Li, Na, K, Cs, Fr, Be, Mg, Ca, Sr, Ba, or Ra.

In a further aspect of the present invention, the hydroxycitric acid is bound to calcium and potassium and derived from a plant of the genus Garcinia or from the plant Garcinia cambogia.

In another aspect of the present invention, approximately 900 milligrams to approximately 4,500 milligrams, and more preferably 2,000 to 3,500 milligrams, and even more preferably 2,700 to 2,800 milligrams of (−) hydroxycitric acid is administered daily.

In yet another aspect of the present invention, the hydroxycitric acid is administered orally, three times a day in three substantially equally divided doses, approximately 30 to 60 minutes before the person or other mammal consumes a meal.

In yet another aspect on the present invention, the composition administered further comprises one or more of the following: gymnemic acid, green tea extract and/or chromium.

In yet another aspect on the present invention, the composition administered comprises niacin-bound chromium.

In yet another aspect on the present invention, the composition administered comprises gymnemic acid derived from a plant of the genus Gymnema or the plant Gymnema sylvestre.

In yet another aspect on the present invention, the green tea extract of the composition is comprised of one or more of the following: epigallocatchin gallate, caffeine and theanine.

In yet another aspect on the present invention, the method comprises administering approximately 10 milligrams to approximately 1,000 milligrams of gymnemic acid daily, approximately 20 milligrams to 2000 milligrams of green tea extract daily and/or approximately 10 micrograms to approximately 1,000 micrograms of chromium daily.

In yet another aspect on the present invention, the method comprises administering approximately 400 micrograms of chromium, approximately 100 milligrams of gymnemic acid and/or approximately 400 milligrams of epigallocatechin gallate.

In another embodiment of the present invention, a composition for decreasing ghrelin levels in a person or other mammal comprising (−)hydroxycitric acid in an amount sufficient to decrease the ghrelin levels in the person or other mammal is provided.

In another aspect of an embodiment of the present invention, the hydroxycitric acid of the composition is derived from a plant of the genus Garcinia or from the plant Garcinia cambogia or is bound to calcium and potassium.

In a further aspect of an embodiment of the present invention, the hydroxycitric acid of the composition is bound to one or more metals to form a single, double or triple salt

In a further aspect of an embodiment of the present invention, the metal or metals the hydroxycitric acid is bound to from a single double or triple salt are Li, Na, K, Cs, Fr, Be, Mg, Ca, Sr, Ba, or Ra.

In another aspect of an embodiment of the present invention, approximately 900 to approximately 4,500 milligrams, and more preferably 2,000 to 3,500 milligrams and even more preferably 2,700 milligrams to approximately 2,800 milligrams, of the composition is taken by the person or other mammal daily.

In yet another aspect of an embodiment of the present invention, the composition further comprises one or more of the following: gymnemic acid, green tea extract and chromium.

In yet another aspect of an embodiment of the present invention, the composition further comprises niacin-bound chromium.

In yet another aspect of an embodiment of the present invention, the hydroxycitric acid of the composition is derived from a plant of the genus Gymnema or the Gymnema sylvestre plant.

In yet another aspect of an embodiment of the present invention, the green tree extract is comprised of one or more of the following: epidallocatchin gallate, caffeine and/or theanine.

In yet another aspect of an embodiment of the present invention, the composition further comprises approximately 10 to 1000 milligrams of gymnemic acid, approximately 20 to 200 milligrams of green tea extract and/or approximately 10 to 1000 micrograms of chromium.

In yet another aspect of an embodiment of the present invention, the composition further comprises approximately 400 micrograms of chromium, approximately 100 milligrams of gymnemic acid and/or approximately 400 milligrams of epigallocatechin gallate.

Other features and advantages of the present invention should become apparent from the following description of the preferred embodiment, taken in conjunction with the accompanying drawings, which illustrate, by way of example, the principles of the invention.

### BRIEF DESCRIPTION OF THE DRAWINGS

FIG. 1 is a chart showing how administering two compositions; (1) HCA-SX and (2) HCA-SX, chromium and gymnemic acid, which incorporate hydroxycitric acid, effects various factors of a person, in accordance with the present invention.

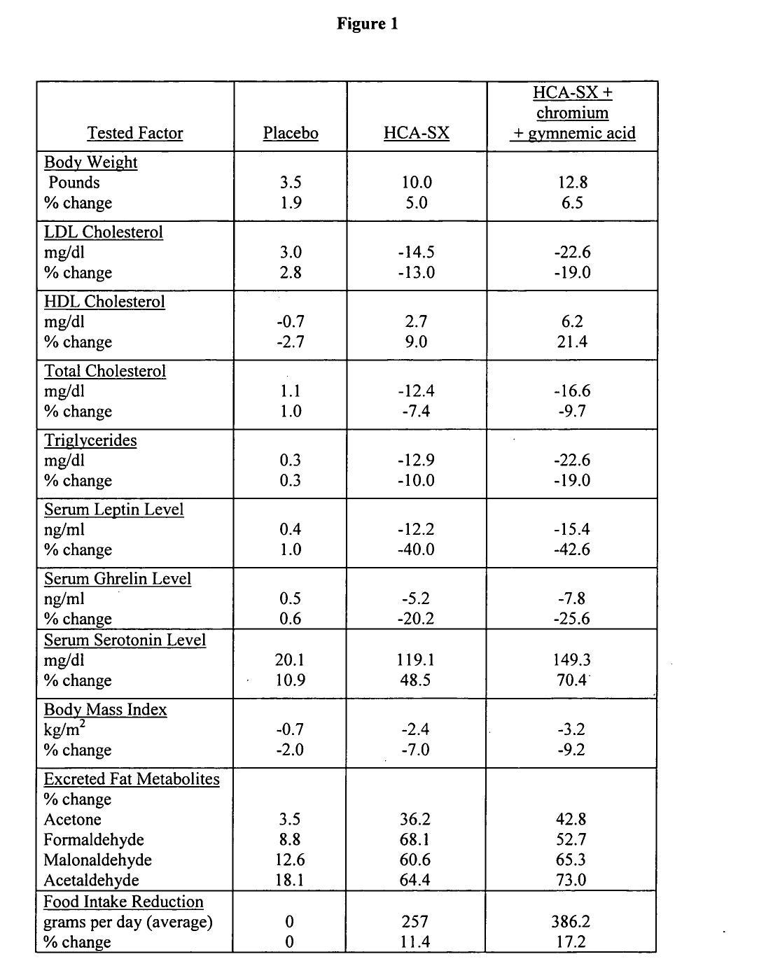

### DETAILED DESCRIPTION OF THE PREFERRED EMBODIMENT

It has been shown that ingestion of compositions incorporating a salt of (−) hydroxycitric acid (HCA) leads to increased levels of serotonin. Serotonin levels, like ghrelin levels, also are related to NPY, and an increase in serotonin levels is a well-established mechanism of appetite suppression. Ingestion of HCA by persons and rodents results in a decrease in serum leptin and ghrelin levels and an increase in serum serotonin levels. This ingestion also leads to a time-dependent decrease in appetite in persons, as shown by measurement of the amount of food remaining on the plates of persons involved in controlled trials.

A preferred known composition incorporating HCA is HCA-SX. HCA-SX is a highly soluble, non-hygroscopic, low-sodium, potassium/calcium salt of (−)hydroxycitric acid extract from the dried fruit rind of Garcinia cambogia containing approximately 60% (−)hydroxycitric acid, marketed by InterHealth Nutraceuticals of Benicia, Calif. This extract is highly soluble in water, and it is readily absorbed and retained by persons. Studies have shown that blood levels of the extract increase for at least 2 hours and remained in the blood for more than 4 to 9 hours after ingestion. Studies also show that eating a meal shortly after consuming the extract reduced its absorption by about 60%. Thus, it is recommended that compositions containing the extract be taken at least 30 to 60 minutes before meals to provide maximum absorption of the HCA. In addition to HCA, decrease in ghrelin levels can be further observed in compositions also incorporating chromium, particularly niacin-bound chromium, gymnemic acid, green tea extract, or any combinations of these. Each of these is discussed below.

Chromium is an essential trace element required for normal protein, fat and carbohydrate metabolism. Chromium levels are known to decrease with age, and marginal chromium deficiencies appear to be widespread. Chromium is important for energy production and plays a role in regulating appetite, reducing sugar cravings and increasing lean body mass. Chromium helps insulin metabolize fat, turn protein into muscle and convert sugar into energy. Chromium has been shown to reduce levels of harmful LDL cholesterol, a form of cholesterol linked to heart disease, and increase levels of beneficial HDL cholesterol. Dietary trends that show increased consumption of more highly processed foods may lead to deficiencies of chromium in persons. Chromium potentiates the action of insulin in vitro and in vivo.

Maximal in vitro activity of chromium requires a special chemical form termed Glucose Tolerance Factor (GTF). GTF is a chromium-nicotinic acid (i.e., niacin) complex and is described in, for example, U.S. Pat. Nos. 4,923,855, 4,954,492 and 5,194,615, all to Jensen and herein incorporated by reference. Chromium extracted from Brewers yeast, which is in the GTF form, is absorbed better than inorganic chromium. GTF is transported across the placental barrier, has different tissue distribution from that of inorganic chromium, and has access to the body pool of chromium that responds to increases in blood insulin. The biologically active form of chromium (GTF) is an essential dietary agent that potentiates the action of insulin and thereby functions in regulating protein, fat and carbohydrate metabolism. A particular form of GTF chromium, marketed under the name ChromeMate® by InterHealth Nutraceuticals, is a unique form of niacin-bound chromium (called chromium nicotinate or polynicotinate) that dramatically increases the effectiveness of chromium in the effects discussed above. Normally, chromium is poorly absorbed and utilized by the body. However, researchers have found that the most potent form of chromium in nature (i.e., the form that best activates insulin) is bound to the B vitamin niacin. In particular researchers have found that a patented oxygen-coordinated chromium-niacin complex is the most potent form of all, being over 18-times more potent than the next closest form of niacin-bound chromium tested. This oxygen-coordinated complex is characterized by chromium bound to an oxygen atom of the carboxylic acid group attached to niacin's pyridine ring structure.

As discussed above, chromium has been shown to reduce LDL cholesterol levels. In particular, administration of this oxygen-coordinated niacin-bound chromium complex (also designated O-NBC) in sufficient amounts has been shown to reduce LDL cholesterol in humans by an average of 14%. Researchers also have shown that O-NBC is significantly more bioavailable than chromium picolinate and chromium chloride. Supplementation with O-NBC therefore has been shown to ameliorate type II diabetes, reduce hypertension, decrease fat mass, and increase lean body mass, as well as help reduce body weight in persons consuming O-NBC. Additionally, high doses of O-NBC have been shown to be completely safe and non-toxic. In contrast, chromium picolinate has been shown to damage DNA and be mutagenic.

Gymnemic acid is found in Gymnema sylvestre, a traditional Ayurvedic herb known to play a role in weight control, by helping to promote normal blood sugar levels and reduce sugar cravings. The active ingredients, gymnemic acid and gurmarin, have similar molecular structures to glucose and provide many benefits to humans and other mammals. Gurmarin has the ability to fill taste bud receptors and reduce the sweet taste of sugary foods, which greatly reduces a person's craving for sweets. Gymnemic acid helps increase the production of insulin by stimulating the production of new insulin-promoting “beta-cells” cells in the pancreas. Gymnemic acid also facilitates insulin release from the beta-cells into the blood stream by increasing beta-cell membrane permeability, and inhibits the absorption of sugar molecules in the intestines during digestion, thus reducing increases in blood sugar levels. Finally, Gymnemic acid has also been shown to lower cholesterol in animal models.

Green tea extract is a bioflavonoid-rich extract having antioxidant properties that contains epigallocatechin gallate (EGCG), caffeine and theanine. EGCG is a potent antioxidant that protects carcinogens, and has been show to reduce cholesterol levels, and blocks the attachment of the bacteria associated with dental cavities to the teeth. Green tea extract also is known for its thermogenic properties that improve fat oxidation, by increasing the metabolic rate of a person consuming it. Caffeine works as a stimulant and aids in fat oxidation, while theanine is known to counter the stimulant effect of caffeine.

The present invention resides in a method and composition for decreasing ghrelin levels in persons who can benefit from such a decrease. The method includes administering to a person or other mammal a composition comprising (−)hydroxycitric acid (HCA) in an amount sufficient to decrease ghrelin levels in that person or other mammal. The composition of the present invention comprises (−)hydroxycitric acid (HCA) in an amount sufficient to decrease ghrelin levels in a person who can benefit from the decrease. The method and composition of the present invention provides the additional advantage of alleviating symptoms in human and other mammals and improving other health related factors.

In a preferred method and composition of the present invention, the composition incorporates a salt of HCA and decreases feeling of hunger in the person or other mammal to which it is administered. In another preferred method and composition, the composition also incorporates one or more of the following: (1), chromium preferably as niacin-bound chromium, Gymnema sylvestre, and green tea extract. Preferably, the method and composition involves a composition of approximately 2,700 mg of HCA daily. As discussed above, one preferred administration of the composition is orally, in three equally divided daily doses roughly 30 to 60 minutes before meals. It should be appreciated that the composition can also can include inert ingredients, fillers or diluents, such as sugar or other known ingredients commonly used in food products. The composition may be in various forms commonly used for dietary supplements, including pill, tablet, capsule, lozenge, gum, liquid, as well as food and beverage products. It should also be appreciated that, in accordance with the present invention, the dosage of the composition can vary significantly based on the weight of the person or other mammal taking the composition, which could be a small child or a morbidly obese adult.

Although the invention has been disclosed in detail with reference only to the preferred embodiments, those skilled in the art will appreciate that additional methods and compositions can be used and made without departing from the scope of the invention. Accordingly, the invention is defined only by the claims set forth below.

We claim:

1. A method for decreasing ghrelin levels in a person or other mammal in need thereof comprising:
administering to a person or other mammal a composition comprised of hydroxycitric acid in an amount sufficient to decrease ghrelin levels in the person or other mammal.

2. A method as defined in claim 1, wherein the hydroxycitric acid is bound to a group IA or group IIA metal to form a single salt.

3. A method as defined in claim 2, wherein the single salt is formed by combining hydroxycitric acid with Li, Na, K, Cs, Fr, Be, Mg, Ca, Sr, Ba, or Ra.

4. A method as defined in claim 1, wherein the hydroxycitric acid is bound to group IA or group IIA metals to form a double salt.

5. A method as defined in claim 4, wherein the double salt is formed by combining hydroxycitric acid with one or more of the following metals: Li, Na, K, Cs, Fr, Be, Mg, Ca, Sr, Ba, or Ra.

6. A method as defined in claim 1, wherein the hydroxycitric acid is bound to a group IA or group IIA metal to form a triple salt.

7. A method as defined in claim 6, wherein the triple salt is formed by combining hydroxycitric acid with one or more of the following metals: Li, Na, K, Cs, Fr, Be, Mg, Ca, Sr, Ba, or Ra.

8. A method as defined in claim 1, wherein the hydroxycitric acid is bound to calcium and potassium.

9. A method as defined in claim 1, wherein the hydroxycitric acid is derived from a plant of the genus Garcinia.

10. A method as defined in claim 1, wherein the hydroxycitric acid is derived from the plant Garcinia cambogia.

11. A method as defined in claim 1, wherein hydroxycitric acid administered is approximately 900 milligrams to approximately 4,500 milligrams daily.

12. A method as defined in claim 2, wherein hydroxycitric acid administered is approximately 900 milligrams to approximately 4,500 milligrams daily.

13. A method as defined in claims 4, wherein hydroxycitric acid administered is approximately 900 milligrams to approximately 4,500 milligrams daily.

14. A method as defined in claims 6, wherein hydroxycitric acid administered is approximately 900 milligrams to approximately 4,500 milligrams daily.

15. A method as defined in claims 8, wherein hydroxycitric acid administered is approximately 900 milligrams to approximately 4,500 milligrams daily.

16. A method as defined in claims 9, wherein hydroxycitric acid administered is approximately 900 milligrams to approximately 4,500 milligrams daily.

17. A method as defined in claims 10, wherein hydroxycitric acid administered is approximately 900 milligrams to approximately 4,500 milligrams daily.

18. A method as defined in claims 1, wherein hydroxycitric acid administered is approximately 2,000 milligrams to approximately 3,500 milligrams daily.

19. A method as defined in claims 2, wherein hydroxycitric acid administered is approximately 2,000 milligrams to approximately 3,500 milligrams daily.

20. A method as defined in claims 4, wherein hydroxycitric acid administered is approximately 2,000 milligrams to approximately 3,500 milligrams daily.

21. A method as defined in claims 6, wherein hydroxycitric acid administered is approximately 2,000 milligrams to approximately 3,500 milligrams daily.

22. A method as defined in claims 7, wherein hydroxycitric acid administered is approximately 2,000 milligrams to approximately 3,500 milligrams daily.

23. A method as defined in claims 8, wherein hydroxycitric acid administered is approximately 2,000 milligrams to approximately 3,500 milligrams daily.

24. A method as defined in claims 9, wherein hydroxycitric acid administered is approximately 2,000 milligrams to approximately 3,500 milligrams daily.

25. A method as defined in claim 1, wherein the hydrbxycitric acid administered is approximately 2,700 milligrams to approximately 2,800 milligrams daily.

26. A method as defined in claim 2, wherein the hydroxycitric acid administered is approximately 2,700 milligrams to approximately 2,800 milligrams daily.

27. A method as defined in claim 4, wherein the hydroxycitric acid administered is approximately 2,700 milligrams to approximately 2,800 milligrams daily.

28. A method as defined in claim 6, wherein the hydroxycitric acid administered is approximately 2,700 milligrams to approximately 2,800 milligrams daily.

29. A method as defined in claim 7, wherein the hydroxycitric acid administered is approximately 2,700 milligrams to approximately 2,800 milligrams daily.

30. A method as defined in claim 8, wherein the hydroxycitric acid administered is approximately 2,700 milligrams to approximately 2,800 milligrams daily.

31. A method as defined in claim 9, wherein the hydroxycitric acid administered is approximately 2,700 milligrams to approximately 2,800 milligrams daily.

32. A method as defined in claim 10, wherein the hydroxycitric acid administered is administered in three substantially equally divided doses three times a day, approximately 30 to 60 minutes before the person or other mammal consumes a meal.

33. A method as defined in claim 11, wherein the hydroxycitric acid administered is administered in three substantially equally divided doses three times a day, approximately 30 to 60 minutes before the person or other mammal consumes a meal.

34. A method as defined in claim 25, wherein the hydroxycitric acid administered is administered in three substantially equally divided doses three times a day, approximately 30 to 60 minutes before the person or other mammal consumes a meal.

35. A method as defined in claim 34, wherein the hydroxycitric acid is bound to calcium and potassium.

36. A method as defined in claim 1, wherein the composition further comprises one or more of the following: gymnemic acid, green tea extract, and chromium.

37. A method as defined in claim 36, wherein, if the composition comprises chromium, the chromium administered is niacin-bound chromium.

38. A method as defined in claim 36, wherein, if the composition comprises gymnemic acid, the gymnemic acid administered is derived from a plant of the genus Gymnema.

39. A method as defined in claim 36, wherein, if the composition comprises gymnemic acid, the gymnemic acid administered is derived from Gymnema sylvestre.

40. A method as defined in claim 36, wherein, if the composition comprises green tea extract, the green tree extract administered comprises one or more of the following: epigallocatchin gallate, caffeine and theanine.

41. A method as defined in claim 36, wherein, if the composition comprises gymnemic acid, the gymnemic acid administered is approximately 10 milligrams to approximately 1,000 milligrams daily, if the composition comprises green tea extract, the green tea extract administered is approximately 20 milligrams to 2000 milligrams daily and, if the composition comprises chromium, the chromium administered is approximately 10 micrograms to approximately 1,000 micrograms daily.

42. A method as defined in claim 11, wherein composition further comprises one or more of the following: approximately 400 micrograms of chromium, approximately 100 milligrams of gymnemic acid and approximately 400 milligrams of epigallocatechin gallate.

43. A method of reducing and maintaining body weight in a person or other mammal in need thereof by decreasing ghrelin levels in the person or other mammal comprising:
administering to a person or other mammal in need of reducing or maintaining body weight a composition comprised of hydroxycitric acid in an amount sufficient to decrease ghrelin levels in the person or other mammal.

44. A method as defined in claim 43, wherein the hydroxycitric acid is bound to a group IA or group IIA metal to form a single salt.

45. A method as defined in claim 44, wherein the single salt is formed by combining hydroxycitric acid with Li, Na, K, Cs, Fr, Be, Mg, Ca, Sr, Ba, or Ra.

46. A method as defined in claim 43, wherein the hydroxycitric acid is bound to group IA or group IIA metals to form a double salt.

47. A method as defined in claim 46, wherein the double salt is formed by combining hydroxycitric acid with one or more of the following metals: Li, Na, K, Cs, Fr, Be, Mg, Ca, Sr, Ba, or Ra.

48. A method as defined in claim 43, wherein the hydroxycitric acid is bound to a group IA or group IIA metal to form a triple salt.

49. A method as defined in claim 48, wherein the double salt is formed by combining hydroxycitric acid with one or more of the following metals: Li, Na, K, Cs, Fr, Be, Mg, Ca, Sr, Ba, or Ra.

50. A method as defined in claim 43, wherein the hydroxycitric acid is bound to calcium and potassium.

51. A method as defined in claim 43, wherein the hydroxycitric acid is derived from a plant of the genus Garcinia.

52. A method as defined in claim 43, wherein the hydroxycitric acid is derived from the plant Garcinia cambogia.

53. A method as defined in claims 43, wherein hydroxycitric acid administered is approximately 900 milligrams to approximately 4,500 milligrams daily.

54. A method as defined in claims 44, wherein hydroxycitric acid administered is approximately 900 milligrams to approximately 4,500 milligrams daily.

55. A method as defined in claims 46, wherein hydroxycitric acid administered is approximately 900 milligrams to approximately 4,500 milligrams daily.

56. A method as defined in claims 48, wherein hydroxycitric acid administered is approximately 900 milligrams to approximately 4,500 milligrams daily.

57. A method as defined in claims 50, wherein hydroxycitric acid administered is approximately 900 milligrams to approximately 4,500 milligrams daily.

58. A method as defined in claims 51, wherein hydroxycitric acid administered is approximately 900 milligrams to approximately 4,500 milligrams daily.

59. A method as defined in claims 52, wherein hydroxycitric acid administered is approximately 900 milligrams to approximately 4,500 milligrams daily.

60. A method as defined in claims 43, wherein hydroxycitric acid administered is approximately 2,000 milligrams to approximately 3,500 milligrams daily.

61. A method as defined in claims 44, wherein hydroxycitric acid administered is approximately 2,000 milligrams to approximately 3,500 milligrams daily.

62. A method as defined in claim 46, wherein the hydroxycitric acid administered is approximately 2,000 milligrams to approximately 3,500 milligrams daily.

63. A method as defined in claim 48, wherein the hydroxycitric acid administered is approximately 2,000 milligrams to approximately 3,500 milligrams daily.

64. A method as defined in claims 50, wherein hydroxycitric acid administered is approximately 2,000 milligrams to approximately 3,500 milligrams daily.

65. A method as defined in claims 50, wherein hydroxycitric acid administered is approximately 2,000 milligrams to approximately 3,500 milligrams daily.

66. A method as defined in claims 52, wherein hydroxycitric acid administered is approximately 2,000 milligrams to approximately 3,500 milligrams daily.

67. A method as defined in claim 43, wherein the hydroxycitric acid administered is approximately 2,700 milligrams to approximately 2,800 milligrams daily.

68. A method as defined in claim 44, wherein the hydroxycitric acid administered is approximately 2,700 milligrams to approximately 2,800 milligrams daily.

69. A method as defined in claim 46, wherein the hydroxycitric acid administered is approximately 2,700 milligrams to approximately 2,800 milligrams daily.

70. A method as defined in claim 48, wherein the hydroxycitric acid administered is approximately 2,700 milligrams to approximately 2,800 milligrams daily.

71. A method as defined in claim 50, wherein the hydroxycitric acid administered is approximately 2,700 milligrams to approximately 2,800 milligrams daily.

72. A method as defined in claim 51, wherein the hydroxycitric acid administered is approximately 2,700 milligrams to approximately 2,800 milligrams daily.

73. A method as defined in claim 52, wherein the hydroxycitric acid administered is approximately 2,700 milligrams to approximately 2,800 milligrams daily.

74. A method as defined in claim 53, wherein the hydroxycitric acid administered is administered in three substantially equally divided doses three times a day, approximately 30 to 60 minutes before the person or other mammal consumes a meal.

75. A method as defined in claim 59, wherein the hydroxycitric acid administered is administered in three substantially equally divided doses three times a day, approximately 30 to 60 minutes before the person or other mammal consumes a meal.

76. A method as defined in claim 67, wherein the hydroxycitric acid administered is administered in three substantially equally divided doses three times a day, approximately 30 to 60 minutes before the person or other mammal consumes a meal.

77. A method as defined in claim 75, wherein the hydroxycitric acid is bound to calcium and potassium.

78. A method as defined in claim 43, wherein the composition further comprises one or more of the following: gymnemic acid, green tea extract, and chromium.

79. A method as defined in claim 78, wherein, if the composition comprises chromium, the chromium administered is niacin-bound chromium.

80. A method as defined in claim 78, wherein, if the composition comprises gymnemic acid, the gymnemic acid administered is derived from a plant of the genus Gymnema.

81. A method as defined in claim 78, wherein, if the composition comprises gymnemic acid, the gymnemic acid administered is derived from Gymnema sylvestre.

82. A method as defined in claim 78, wherein, if the composition comprises green tea extract, the green tree extract administered comprises one or more of the following: epigallocatchin gallate, caffeine and theanine.

83. A method as defined in claim 78, wherein, if the composition comprises gymnemic acid, the gymnemic acid administered is approximately 10 milligrams to approximately 1,000 milligrams daily, if the composition comprises green tea extract, the green tea extract administered is approximately 20 milligrams to 2000 milligrams daily and, if the composition comprises chromium, the chromium administered is approximately 10 micrograms to approximately 1,000 micrograms daily.

84. A method as defined in claim 53, wherein the composition further comprises one or more of the following: approximately 400 micrograms of chromium, approximately 100 milligrams of gymnemic acid and approximately 400 milligrams of epigallocatechin gallate.

85. A composition for decreasing ghrelin levels in a person or other mammal comprising:
hydroxycitric acid in an amount sufficient to decrease the ghrelin levels in the person or other mammal.

86. A composition of claim 85, wherein the hydroxycitric acid is bound to a group IA or group IIA metal to form a single salt.

87. A composition of claim 86, wherein the single salt is formed by combining hydroxycitric acid with Li, Na, K, Cs, Fr, Be, Mg, Ca, Sr, Ba, or Ra.

88. A composition of claim 85, wherein the hydroxycitric acid is bound to group IA or group IIA metals to form a double salt.

89. A composition of claim 88, wherein the double salt is formed by combining hydroxycitric acid with one or more of the following metals: Li, Na, K, Cs, Fr, Be, Mg, Ca, Sr, Ba, or Ra.

90. A composition of claim 85, wherein the hydroxycitric acid is bound to a group IA or group IIA metal to form a triple salt.

91. A composition of claim 90, wherein the triple salt is formed by combining hydroxycitric acid with one or more of the following metals: Li, Na, K, Cs, Fr, Be, Mg, Ca, Sr, Ba, or Ra.

92. A composition of claim 85, wherein the hydroxycitric acid is bound to calcium and potassium.

93. A composition of claim 85, wherein the hydroxycitric acid is derived from a plant of the genus Garcinia.

94. A composition of claim 85, wherein the hydroxycitric acid is derived from the plant Garcinia cambogia.

95. A composition of claim 85, wherein the amount of hydroxycitric acid sufficient to decrease the ghrelin levels is approximately 900 milligrams to approximately 4,500 milligrams.

96. A composition of claim 85, wherein the amount of hydroxycitric acid sufficient to decrease the ghrelin levels is approximately 2,000 milligrams to approximately 3,500 milligrams.

97. A composition of claim 85, wherein the amount of hydroxycitric acid sufficient to decrease the ghrelin levels is approximately 2,700 milligrams to approximately 2,800 milligrams.

98. A composition of claim 85, wherein the composition further comprises one or more of the following: gymnemic acid, green tea extract and chromium.

99. A composition of claim 98, wherein, if the composition comprises chromium, the chromium is niacin-bound chromium.

100. A composition of claim 98, wherein, if the composition comprises gymnemic acid, the gymnemic acid is derived from a plant of the genus Gymnema.

101. A composition of claim 98, wherein, if the composition comprises gymnemic acid, the gymnemic acid is derived from Gymnema sylvestre.

102. A composition of claim 98, wherein, if the composition comprises green tea extract, the green tree extract comprises one or more of the following: epigallocatchin gallate, caffeine and theanine.

103. A composition of claim 98, wherein, if the composition comprises gymnemic acid, it comprises approximately 10 milligrams to approximately 1,000 milligrams of gymnemic acid, if the composition comprises green tea extract, it comprises approximately 20 milligrams to 2000 milligrams of green tea extract and, if the composition comprises chromium, it comprises approximately 10 micrograms to approximately 1,000 micrograms of chromium.

104. A composition of claim 98, wherein composition further comprises one or more of the following: approximately 400 micrograms of chromium, approximately 100 milligrams of gymnemic acid and approximately 400 milligrams of epigallocatechin gallate.
